(19) United States
(12) Patent Application Publication
(10) Pub. No.: US 2003/0211522 A1
(43) Pub. Date: Nov. 13, 2003

(54) Methods for fetal DNA detection and allele quantitation

(76) Inventors: Gregory M. Landes, Livermore, CA (US); Lesley Michalowsky, Sudbury, MA (US); Glenn Miller, Hopkinton, MA (US); William Weber, Franklin, MA (US)

Correspondence Address: Bingham McCutchen LLP, Suite 1800, Three Embarcadero Center, San Francisco, CA, 94111, US

(21) Appl. No.: 10/346,514

(22) Filed: Jan. 17, 2003

Related U.S. Application Data
(60) Provisional application No. 60/349,877, filed on Jan. 18, 2002.

(51) Int. Cl. C12Q 1/68; C12P 19/34

(52) U.S. Cl. 435/6; 435/91.2

(57) ABSTRACT

The present invention provides non-invasive methods to distinguish fetal DNA from maternal DNA and thereby detect fetal aneuploidies and alleles. The methods require isolation of fetal DNA from maternal serum and treatment with a reagent that creates primary sequence differences between maternal and fetal DNA that exhibit differential methylation. Various methods including quantitative PCR is used to identify detect fetal aneuploidies and alleles. In one embodiment, the method is useful to identify imprinting genes in subjects, including adults.

### CROSS-REFERENCE TO RELATED APPLICATIONS

This application claims priority under 35 U.S.C. § 119 (e) to U.S. Provisional Application Serial Nos. 60/349,877, filed Jan. 18, 2002, the contents of which are hereby incorporated by reference into the present disclosure.

### TECHNICAL FIELD

The present invention relates to the fields of molecular biology and genetics and provides methods for prenatal detection of chromosome aberrations and mutations.

### BACKGROUND

Prenatal testing is capable of identifying a variety of serious genetic problems, including chromosomal abnormalities and other disease-related mutations. Typically, such testing is performed on samples of fetal cells obtained, for example, using invasive procedures including amniocentesis, chorionic villus sampling, or fetal blood sampling. The chromosomes within these cells are then analyzed by cytogenesis procedures such as karyotyping by fluorescent in situ hybridization (FISH) using chromosome specific fluorescent probes to detect gross anomalies such as chromosome aneuploidies. Alternatively, specific genetic defects such as point mutations in disease-associated genes can be detected by molecular analyses designed to identify single nucleotide polymorphisms (SNPs) and other small mutations. In either case, the invasive procedures required to obtain these fetal cell samples are less than ideal since they introduce an inherent risk of harming the mother or the fetus, and can cause miscarriage.

The development of a non-invasive prenatal genetic screen would fill a large, umnet need in prenatal healthcare. Despite substantial effort, investment and technical advancements on some fronts, significant challenges exist which have hindered the development of a robust testing platform for prenatal diagnosis. A small number of fetal cells are known to cross the placenta and circulate in maternal blood with estimates ranging from 1 to 2000 fetal cells per mL of blood (Senyei and Wasserman (1993) Obstet. Gynecol. Clin. North Am. 20(3):583-598). Fetal nucleated red blood cells (NRBCs) contain a full genetic complement, are relatively distinct from maternal cells, and have a finite life span. Assays employing FISH and PCR-based techniques have provided diagnostic information on such clinical cell samples, although the ability to reliably demonstrate sufficient numbers of fetal cells for genetic evaluation has not been shown by the scientific community at large (Bianchi (1997) Curr. Opin. Obstet. Gynecol. 9(2): 121 -125). This inconsistency contrasts with technical improvements in platform development, specifically, bioimaging, immunocytochemistry of fetal globin, and FISH. For example, Poon, L. L. M. et al. (2000) Lancet 356:1819-1820, reports that FISH analysis of maternal plasma samples can identify fetal cells with three chromosome-21 signals indicative of a fetus affected by trisomy 21 (Down's Syndrome).

Recent reports indicate that maternal serum or plasma may be a relatively rich source of fetal DNA based on PCR determinations. It has been shown that fetal DNA can be consistently detected in maternal serum as early as 7 weeks, increases in abundance during gestation, and are detectable 1 month but not 2 months postpartum. In ˜100 cases, the lowest fetal DNA concentration in plasma as measured by PCR was greater than 20 fetal cell equivalents per mL of maternal blood with some instances where fetal DNA constituted as much as 5% of the total DNA in plasma. This type of fetal source could enable PCR-based genetic testing if the amplification process can be made fetal-specific or if the fetal amplicons can be discriminated from maternal amplicons by additional steps. Such testing would provide a valuable improvement in existing methods for detecting fetal genetic defects since it would be non-invasive, easy to perform and reproducible. The present invention provides methods for performing such analyses.

### DESCRIPTION OF THE INVENTION

The present invention provides several non-invasive methods for detecting fetal alleles and aneuploidies. DNA is first isolated from maternal serum and treated with a reagent which differentially modifies methylated and non-methylated DNA, e.g., bisulfite. The DNA is amplified using quantitative PCR and primers selected to amplify target sequences on a potentially abnormal chromosome. Control quantitative PCR with a second pre-selected primer is conducted on a non-trisomic chromosome and the ratio of the quantity of the two PCR products are determined, thereby detecting fetal aneuploidies.

In an alternative embodiment, the invention provides a method for detecting fetal chromosome aneuploidies by performing quantitative PCR on bisulfite-treated DNA isolated from maternal serum. Quantitative PCR is performed on the sample with a primer pair homologous to a test chromosome sequence that is differentially methylated in maternal DNA and in fetal DNA, where the primer pair only primes bisulfite treated unmethylated DNA. A “control” quantitative PCR with a primer pair homologous to a control chromosome sequence that is differentially methylated in maternal DNA and in fetal DNA, where the primer pair only primes bisulfite treated unmethylated DNA. The ratio of the quantity of PCR product produced for the test chromosome compared with the control chromosome, thereby detecting fetal aneuploidies.

In a further aspect, alleles of fetal DNA can be detected by treating DNA isolated from maternal serum with bisulfite. PCR is performed with a primer pair that amplifies the gene of interest when it has been modified by bisulfite treatment and analyzing the PCR product to identify the allele. Analysis can be performed by method known in the art, e.g., DNA sequence (Maxam and Gilbert (1980) Methods in Enzymology 65(pt 1):497 and Sanger et al. (1977) Proc. Natl. Acad. Sci. USA 74:5463), DNA microarrays (E. M. Southern (1996) Tr. Genetics 12(3):110-115; Southern, E. M. et al. (1999) Nature Genetics, Supp. 21:5-9; and Hacia, J. G. (1999) Nature Genetics, Supp. (1999) 21:42-47), SSCP (Dean et al. (1990) Cell 61:863; Glavac and Dean (1993) Hum. Mutation 2:404; and Poduslo et al. (1992) Am. J. Hum. Genet. 49:106) and LAMP (U.S. Pat. No. 6,297,010).

Further provided by this invention is a non-invasive method for detecting imprinted genes in a subject (not limited to fetal) by treating the DNA isolated from the subject with bisulfite and performing PCR with a primer pair for a polymorphic region that only amplified bisulfite treated unmethylated DNA. The PCR product is analyzed to identify the polymorphism. Analysis can be performed by method known in the art, e.g., DNA sequence, DNA microarrays, SSCP, LAMP.

### BRIEF DESCRIPTION OF THE FIGURES

FIGS. 1A and 1B graphically show application of the method of this invention to detect fetal alleles.

FIGS. 2A and 2B graphically show application of the method of this invention to detect and quantitate a single base extension.

FIG. 3 graphically shows an embodiment of the method of the invention using semi-quantitative hybridization to compare differentially methylated sites on several alleles.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention employs, unless otherwise indicated, conventional techniques of molecular biology (including recombinant techniques), microbiology, cell biology, biochemistry and immunology, which are within the skill of the art. Such techniques are explained filly in the literature, such as, molecular cloning: a laboratory manual, second edition (Sambrook et al., 1989); current protocols in molecular biology (F. M. Ausubel et al., eds., 1987); oligonucleotide synthesis (M. J. Gait, ed., 1984); animal cell culture (R. I. Freshney, ed., 1987); methods in enzymology (Academic Press, Inc.); handbook of experimental immunology (D. M. Wei & C. C. Blackwell, eds.); gene transfer vectors for mammalian cells (J. M. Miller & M. P. Calos, eds., 1987); pcr:the polymerase chain reaction, (Mullis et al., eds., 1994); current protocols in immunology (J. E. Coligan et al., eds., 1991); anitobodies: a laboratory manual (E. Harlow and D. Lane eds. (1988)); and pcr 2:a practical approach (M. J. MacPherson, B. D. Hames and G. R. Taylor eds. (1995)).

Definitions

As used herein, certain terms may have the following defined meanings.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi- stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The terms “chromosomal abnormalities” and “chromosomal aberrations” are used interchangeably to refer to numerical and structural alterations in a chromosome which give rise to an abnormal or pathological phenotype. Chromosomal abnormalities can be of several types, for example, extra or missing individual chromosomes, extra or missing portions of a chromosome (segmental duplications or deletions), breaks, rings and rearrangements, among others.

Numerical alterations include chromosomal aneuploidies. The term “aneuploidy” refers to the occurrence of at least one more or one less chromosome than the normal diploid number of chromosomes leading to an unbalanced chromosome complement. Chromosomal aneuploidy is associated with a large number of genetic disorders and degenerative diseases. Examples of common aneuploid conditions include Down's syndrome (trisomy 21), Edward syndrome (trisomy 18), Patau syndrome (trisomy 13), Turner syndrome associated with an absence of an X chromosome (XO), Kleinfelter syndrome associated with an extra X chromosome (XXY), XYY syndrome, triple X syndrome, and the like.

The term “antigen” is well understood in the art and includes substances which are immunogenic, i.e., immunogens, as well as substances which induce immunological unresponsiveness, or anergy, i.e., anergens.

A “subject” is a vertebrate, preferably a mammal, more preferably a human. Mammals include, but are not limited to, murines, simians, humans, farm animals, sport animals, and pets.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

The present invention provides a non-invasive method for detecting fetal aneuploidies. DNA is first isolated from maternal serum and treating with a reagent which differentially modifies methylated and non-methylated DNA, e.g., bisulfite.

In general, fetal DNA is hypomethylated relative to adult DNA reflecting transcriptional silencing of specific genes expressed early in development. One means of generating fetal-specific PCR products is to identify loci that are unmethylated in fetal DNA and methylated in adult/maternal DNA. Another means to detect fetal-specific DNA is to identify loci that are methylated in fetal DNA and unmethylated in adult/maternal DNA. Loci of this type are differentially reactive with bisulfite such that unmethylated Cs in DNA undergo oxidative deamination, resulting in C to U transitions. Methylated Cs are not reactive with bisulfite, and consequently, are unaffected. Bisulfite treatment of fetal and maternal DNA present in maternal serum will create primary sequence differences between fetal and maternal loci that exhibit differential methylation. The DNA is amplified using quantitative PCR and primers selected to amplify sequences on a potentially abnormal chromosome. Control quantitative PCR with a second pre-selected primer is conducted on a normal or control chromosome (i.e., a chromosome not having the suspected anomaly) and the ratio of the quantity of the two PCR products are determined, thereby detecting fetal aneuploidies. If the loci of interest are from chromosome 13, 18 or 21, and quantitative PCR strategies are employed, e.g., real-time PCR and chromosome copy number can be determined. Similarly, if the loci are also highly polymorphic such that both alleles can be discerned, chromosome aneuploidy can be readily revealed.

In an alternative embodiment, the invention provides a method for detecting fetal chromosome aneuploidies by treating DNA isolated from maternal serum with bisulfite and then performing quantitative PCR on the sample with a primer pair homologous to a test chromosome sequence that is differentially methylated in maternal DNA and in fetal DNA, where the primer pair only primes bisulfite treated unmethylated DNA. A “control” quantitative PCR is conducted with a primer pair homologous to a control chromosome sequence that is differentially methylated in maternal DNA and in fetal DNA, where the primer pair only primes bisulfite treated unmethylated DNA. The ratio of the quantity of PCR product produced for the test chromosome is compared with the control chromosome, thereby detecting fetal aneuploidies.

Another group (Poon et al. (2002) Clin. Chem. 48(l):35-45) has proposed an approach that is quite different from the subject invention. The authors in the Poon et al. reference rely upon an imprinted locus, where methylation status depends upon whether or not the allele is inherited from the mother or the father. This is quite different from the subject invention, that exploits a more global difference in fetal vs. adult methylation that is not dependent upon parent of origin. An advantage of the subject invention is that it allows all fetal alleles to be analyzed, which is not contemplated by the authors of Poon et al. (2002) supra.

Several methods are known in the art for performing quantitative PCR. Examples of such include, but are not limited to use of a fluorescent probe measured with the ABI PRISM® 7700 Sequence Detection System (Applied Biosystems, Foster City, Calif.), single base extension with a radioactively label nucleotide or single base extension followed by mass spectrometry.

In a further aspect, alleles of fetal DNA can be detected by treating DNA isolated from maternal serum with bisulfite. PCR is performed with a primer pair that amplifies the gene of interest when it has been modified by bisulfite treatment and analyzing the PCR product to identify the allele. See for example, U.S. Pat. No. 5,786,146. Analysis can be performed by method known in the art, e.g., DNA sequence, DNA microarrays, SSCP, LAMP.

Specific examples include mutant alleles that include but are not limited to alpha fetoprotein, globins, sickle cell anemia, β-thalassaemia, Downs syndrome, RhD disease, Duchenne's disease, cystic fibrosis, muscular dystrophy, and Gaucher's syndrome.

Further provided by this invention is a non-invasive method for imprinted genes in a subject (not limited to fetal) by treating the DNA isolated from the subject with bisulfite and performing PCR with a primer pair for a polymorphic region that only amplifies bisulfite treated unmethylated DNA. The PCR product is analyzed to identify the polymorphism. Analysis can be performed by method known in the art, e.g., DNA sequence, DNA microarrays, SSCP, LAMP.

The following examples are intended to illustrate, not limit the invention.

### EXAMPLES

Materials and Methods

Isolation of Fetal DNA from Maternal Blood

Plasma Separation Protocol: Maternal blood is collected into ACDA blood collection tube (Becton Dickinson, Franklin Lakes, NJ) or other appropriate collection tube. The blood is transferred to a fresh, labeled 15 ml conical tube and centrifuged at 600×g for 10 minutes. The clear plasma is removed above the red cell pellet using a 10 ml pipette and transferred to another fresh, labeled 15 ml conical tube. Plasma is centrifuged at 1500×g for 10 minutes and transferred to a fresh, labeled conical tube and stored at −80° C. until DNA isolation.

DNA Isolation Protocol: DNA can be isolated using the commercially available QIAamp® DNA Blood Mini Kit (Qiagen, Hilden, Germany). The kit provides the following reagents: Buffer AL (lysis), Buffer AW1 and AW2 (wash buffer) and Buffer AE (elution). Prior to running the procedure the following preliminary steps are required: 1) equilibrate samples to room temperature; 2) thaw Proteinase K at room temperature; 3) turn on WPCR heat-block to 56° C.; 4) equilibrate buffer AW1 and Buffer AW2, if precipitate has formed in Buffer AL, dissolve by incubating at 70° C. All centrifugation steps are carried out at room temperature.

About 20 μl of RNase A (100 mg/ml) is added to the bottom of a 1.5 ml micro-centrifuge tube. About 200 μl of plasma sample is added to the micro-centrifuge tube and mixed well by pipetting up and down. If the sample volume is less than 200 μl, add the appropriate volume of PBS to bring it up to 200 μl. If the sample volume is more than 200 μl, prepare multiple tubes of 200 μl sample in each. Load column successively and save.

Add 20 μl of 20 mg/ml Proteinase K and mix well by pipetting up and down. Then add 200 μl Buffer AL to the sample and mix thoroughly by pulse-vortexing for 50 seconds. The sample is then incubated at 56° C. for 10 minutes. Briefly centrifuge the 1.5 ml micro-centrifuge to remove drops from inside the lid. 200 μl of 100% ethanol is added to the sample and mixed by pulse-vortexing for 15 seconds. After mixing, centrifuge the sample to remove drops from inside the lid.

This mixture is added to the loaded column (see above) in a 2 ml collection tube without wetting the rim. The tube is centrifuged at 6000×g (8000 rpm) for 1 minute. Place the spin column in a clean 2 ml collection tube. Centrifugation at 6000×g (8000 rpm) is sufficient to pull most plasma samples through the column. 500 μl Buffer AW1 is then added without wetting the rim, and centrifuged at 20,000×g (14000 rpm) for 3 minutes. The spin column is placed in a new 2 ml collection tube and spun again at full speed.

The spin column is then placed in a clean, labeled 1.5 ml micro-centrifuge tube, 50 μl of 56° C. Buffer AE is added to the center of the column and then incubated at 56° C. (heat-block) for 5 minutes. Following incubation, the column is centrifuged at 6000×g (8000 rpm) for 1 minute.

Another 50 μl of 56° C. Buffer AE is added to the center of the column and incubation and centrifugation are repeated, as above.

Bisulfite Treatment of DNA

Protocol A: Sample DNA is sheared or restriction digested (if using less than 1 μg of DNA, 1 μg of yeast tRNA or 1 μg of salmon sperm DNA can be used as a carrier). DNA is denatured with 0.3 M NaOH for 15 minutes at 37° C. and then modified with 5.36 M urea, 3.44 M sodium bisulfite, and 0.5 mM hydroquinone (adjusted to pH 5.0 with NaOH) for 15 hours at 55° C. The samples are overlayed with 100 μl of mineral oil during the incubation. The modified DNA is desalted with the Wizard® DNA clean up kit (Promega. Madison, Wis.) following manufacturer's instructions. DNA is eluted in 50 μl of TE. Free bisulfite is removed by incubating the desalted modified DNA with 0.3 M NaOH for 15 minutes at 37° C. The samples are neutralized by adding NH₄OAc, pH 7.0 to 3 M. The DNA is ethanol precipitated and resuspended in 100 μl TE. Store at −20° C.

Protocol B: Fresh 4M sodium bisulfite and 100 mM hydroquinone is prepared. Sodium bisulfite is prepared by adding 1.6 g sodium bisulfite in 3 ml HPLC H₂0. Adjust to pH 5 with approximately 160 μl of 5 M NaOH. Adjust total volume to 4 ml. Hydroquinone is prepared by adding 0.11 g to 9 ml (for 100 mM). Adjust to pH 5 with NaOH. Adjust to 10 ml total volume.

Unsheared DNA is denatured at 95° C. for 5 minutes. (If less than 1 μg of DNA is used, 1 μg salmon sperm DNA can be used as a carrier). The DNA sample is placed on ice and quickly centrifuged. 5 M NaOH is added to the sample to a final concentration of 0.3 M in a total volume of 100 μl and incubated at 37° C. for 30 minutes.

4 M sodium bisulfite and 100 mM hydroquinone are then added to final concentrations of 3.1 M and 0.5 mM, respectively, pH 5 in a total volume of 500 μl. The sample is then overlaid with 100 μl of mineral oil and incubated at 55° C. for 16 hours.

The sample is desalted with the Wizard® Clean up kit (Promega, Madison, Wis.) according to manufacturer's instructions (i.e., elute in 100 μl, yield about 96 μl). 5 M NaOH is added to a final concentration of 0.3 M in a total volume of 100 μl and the sample is then incubated at 37° C. for 15 minutes.

The DNA is neutralized with 60 μl of 10 M NH₄OAc (final concentration approximately 3 M) and 40 μl HPLC H₂O. The DNA is precipitated by adding 800 μl of cold 96% ethanol, storing at −20° C. for 30 minutes; and centrifuging for 30 minutes at 14,000×g at 4° C.; removing the supernatant and resuspending the pellet in 70% cold ethanol and re-centrifuging for 30 minutes at 4° C. The 70% ethanol wash is repeated and all residual ethanol is removed.

The DNA is resuspended in 25 μl of 0.1×TE.

Protocol C: Fresh 4 M sodium bisulfite and 100 mM hydroquinone is prepared. Sodium bisulfite is prepared by adding 1.6 g sodium bisulfite in 3 ml HPLC H₂0. Adjust to pH 5 with approximately 160 μl of 5 M NaOH. Adjust total volume to 4 ml. Hydroquinone is prepared by adding 0.11 g to 9 ml (for 100 mM). Adjust to pH 5 with NaOH. Adjust to 10 ml total volume.

Unsheared DNA is denatured at 95° C. for 5 minutes. (If less than 1 μg of DNA is used, 1 μg glycogen can be added as a carrier). The DNA is placed on ice and quickly centrifuged. 5 M NaOH is added for a final concentration of 0.3 M in a total volume of 100 μl and incubated at 37° C. for 30 minutes. 4M sodium bisulfite and 100 mM hydroquinone are added to final concentrations of 3.1 M and 0.5 mM, respectively, pH 5, final in a total volume of 500 μl. The sample is overlaid with 100 μl of mineral oil and incubated at 55° C. for 16 hours.

The sample is desalted with the QIA®quick PCR purification kit (Qiagen, Hilden, Germany) according to manufacturer's instructions (i.e., elute in 100 μl, yield about 96 μl). 5 M NaOH is added to a final concentration of 0.3 M in a total volume of 100 μl and the sample is then incubated at 37° C. for 15 minutes.

The DNA is neutralized with 60 μl of 10 M NH₄OAc (final approximately 3 M) and 40 μl HPLC H₂0 and cleaned up with QIA®quick PCR purification kit (Qiagen, Hilden, Germany) according to manufacturer's instructions. Elute in 28 μl yields about 25 μl.

Protocol D: DNA is bisulfite treated using CpGenome™ DNA Modification kit (Intergen Co., Purchase, N.Y.) using the manufacturer's instructions. Briefly, DNA is denatured in NaOH and methylated sites are modified with a solution of bisulfite and hydroquinone. DNA is desalted and cleaned up and treated with alkali to remove free bisulfite. Ammonium acetate is added to neutralize. DNA is ethanol precipitated and cleaned up.

Quantitative PCR: This procedure is accomplished using methods well known in the art, for example, using the procedure of Nuovo, G. J. et al. (1999) J. Histochem. & Cytochem. 47(3):273-279. In this method, any target-specific primer pair is used in combination with a universal energy transfer-labeled primer. UniPrimer-based in situ PCR allows rapid and simple detection of any DNA or RNA target without concern for the background from DNA repair invariably evident in paraffin-embedded tissue when a labeled nucleotide is used.

Alternative procedures are reported in Pertl, B. et al. (1999) Hum. Gen. 98:55-59 and (1999) J. Med. Genet. 36:300-303 as well as Cirigliano, V. et al. (1999) Prenat. Diagn. 19:1099-1103.

Primer Sequences for Detection of Aneuploidies or Disease Genes: Several primer sequences have been demonstrated for detection of aneuploidies or disease genes. Findlay, I. et al. (1998) J. Clin. Pathl: Mol. Pathol. 51:164-167 discloses several primers for the detection of Down's syndrome. Cheung, M-C. et al. (1996) Nature Gen. 14:264-268 discloses primers for amplification of the sickle cell anemia and β-thalassaemia. Sekizawa, A. et al. (1996) Neurology 46:1350 provides several primers for amplification of marker DNA for Duchenne's disease. Sekizawa, A. et al. (1996) Obstet. Gynecol. 87:501 discloses primers for amplification of marker DNA for RhD disease.

### EXPERIMENTAL EXAMPLES

### Example 1

### Bisulfite Treatment and Quantitative PCR

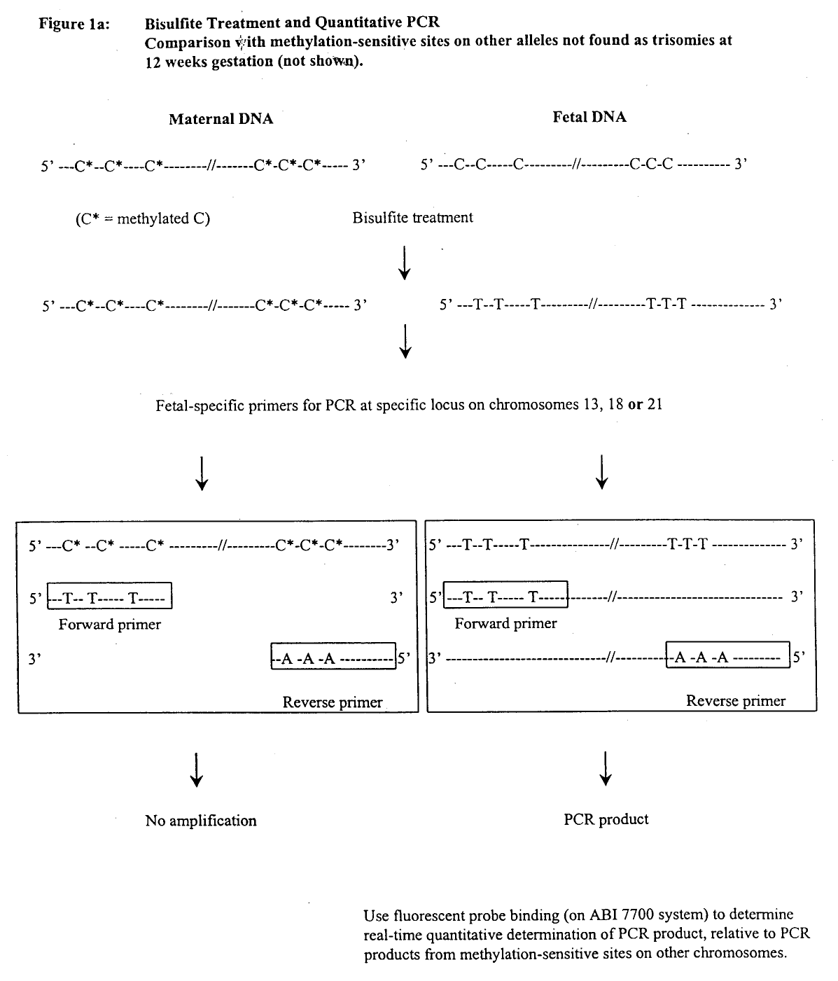

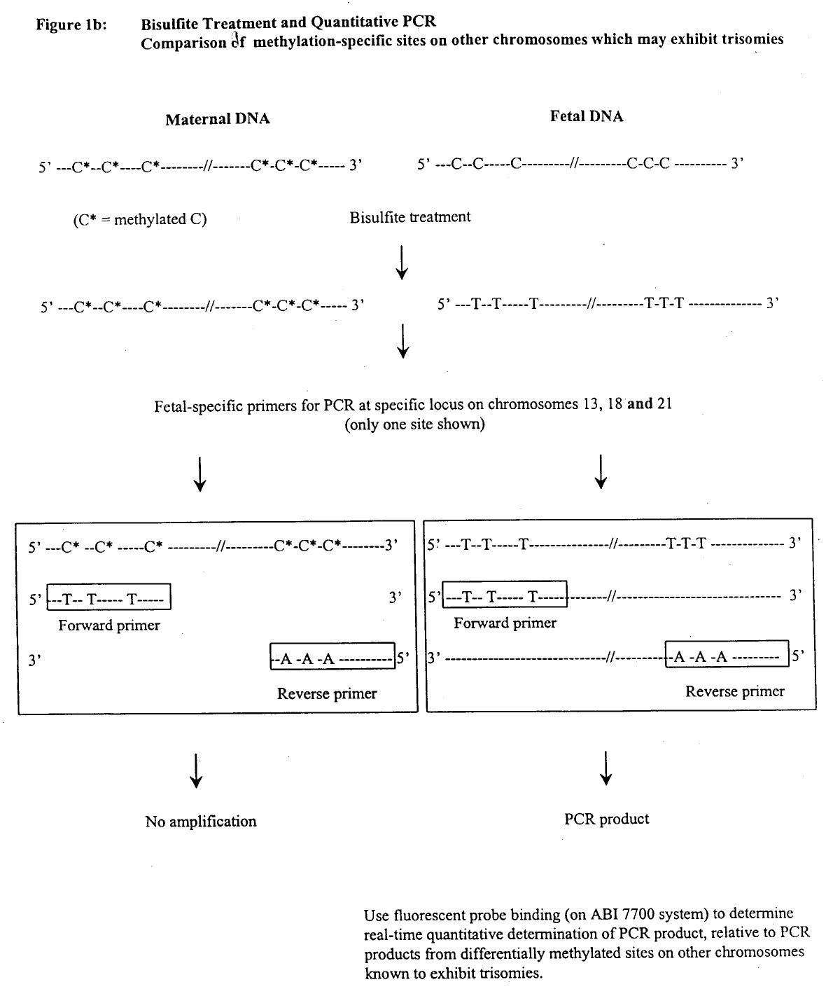

FIG. 1A shows a specific example of application of the method of this invention to identify fetal allele detection. Methylation-specific sites are compared on other alleles, e.g., Chromosome 16, since aneuploidies on this chromosome are early lethal.

FIG. 1B shows a specific example of application of the method of this invention to identify fetal allele detection. Methylation-specific sites are compared to sites on other chromosomes that may exhibit aneuploidies.

### Example 2

### Bisulfite Treatment and Quantitative Single Base Extension

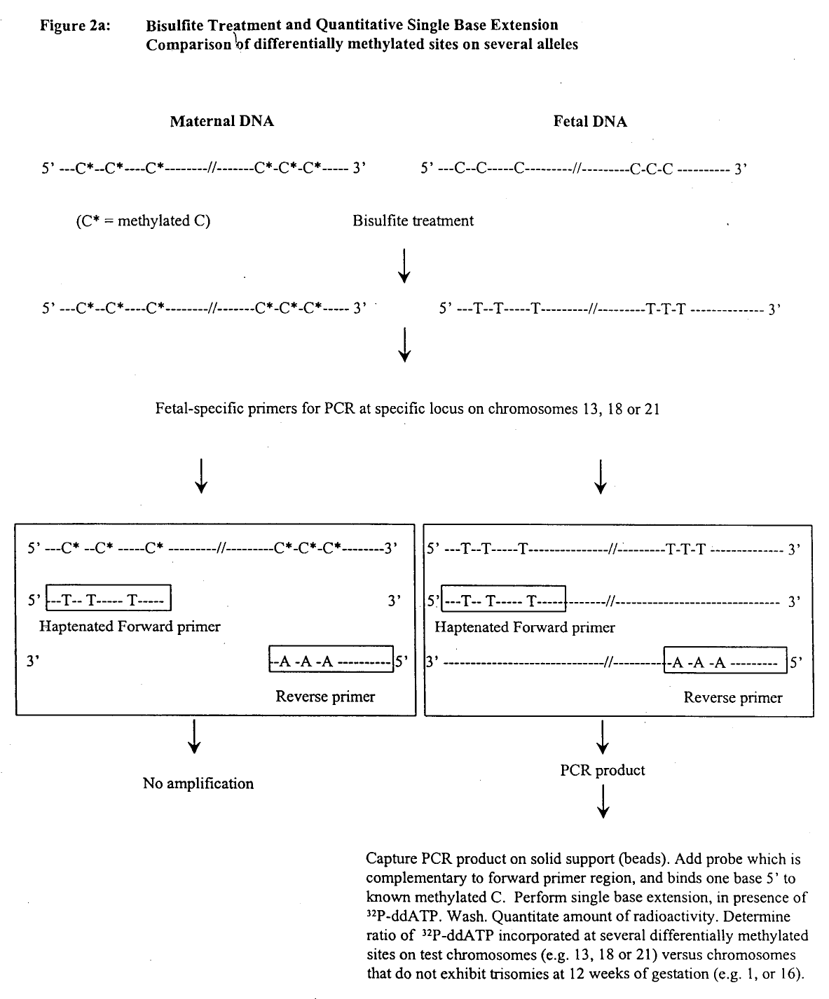

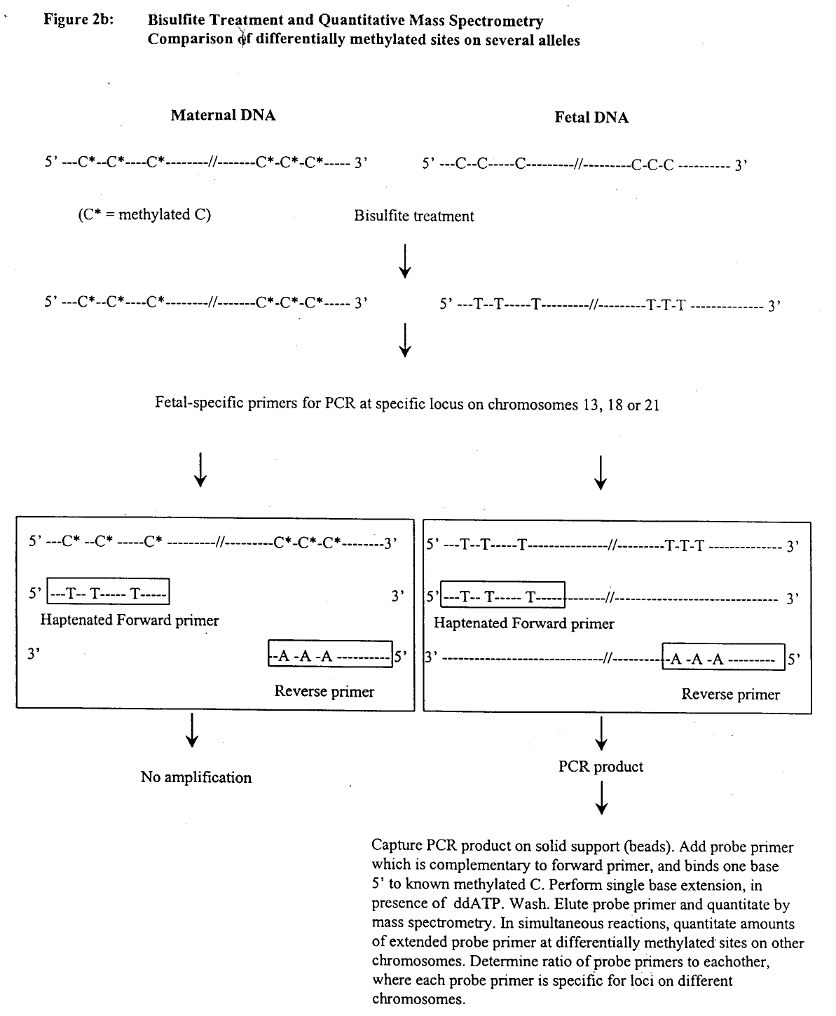

FIG. 2A shows a specific example of the method of this invention wherein differentially methylated sites on several alleles are compared. The DNA is capture PCR'd on a solid support such as beads. A probe which is complementary to forward primer region and binds one base 5′ to known methylated Cysteine (C) is added. Single base extension is performed in the presence of ³²P-ddATP incorporated at several differentially methylated sites on test chromosomes (e.g., 13, 18 or 21) versus chromosomes that do not exhibit aneuploidies at 12 weeks gestation (e.g., 1 or 16).

FIG. 2B shows a specific example of the method of this invention wherein differentially methylated sites on several alleles are compared using bisulfite treatment and quantitative mass spectrometry. The DNA is capture PCR'd on a solid support such as beads. A probe which is complementary to a forward primer region and binds one base 5′ to known methylated Cysteine (C) is added. Single base extension is performed in the presence of ³²P-ddATP. Wash and elute probe primer and quantitate by mass spectrometry. In simultaneous reactions, quantitate amount of extended probe primer at differentially methylated sites on other chromosomes. The ratio of probe primers is determined relative to each other, where each probe primer is specific for loci on different chromosome.

### Example 3

### Bisulfite Treatment and Semi-Quantitative Hybridization

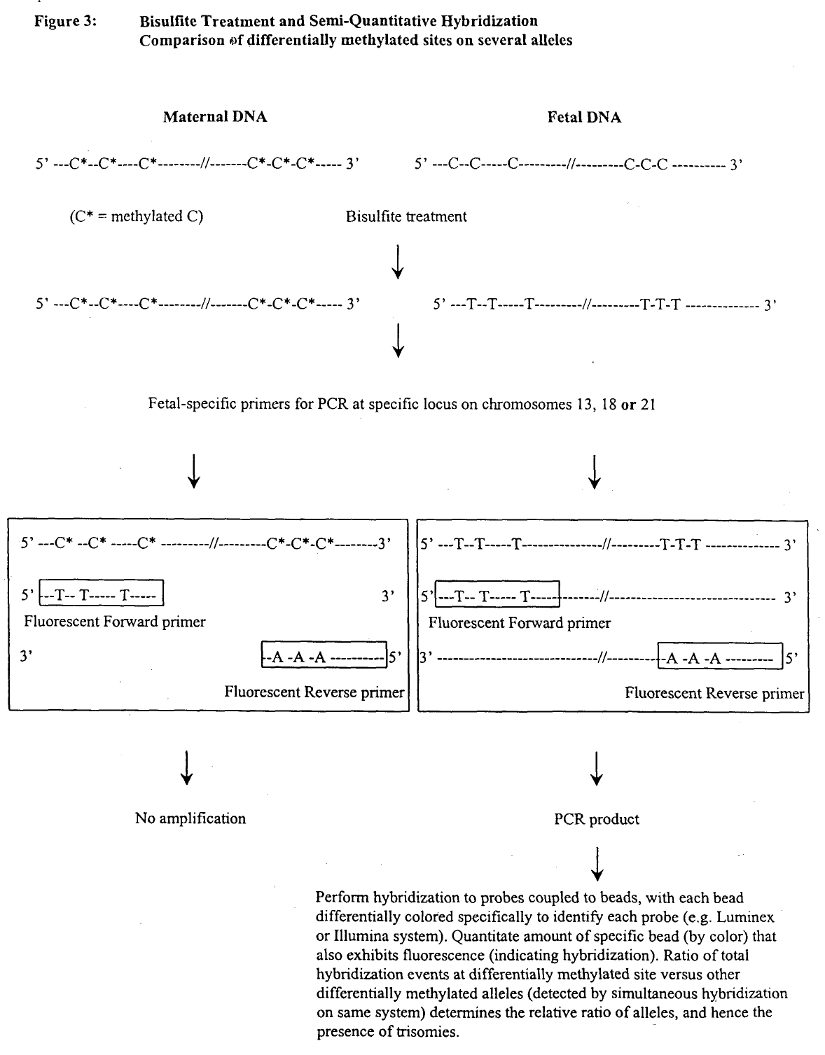

FIG. 3 shows a specific example of the method of this invention wherein differentially methylated sites on several alleles are compared using bisulfite treatment and semi-quantitative hybridization. Hybridization is performed on probes coupled to beads, with each bead differentially colored specifically to identify each probe. High throughput technology platforms useful for such analysis are known in the art and include, for example, microsphere array analysis systems e.g., LabMAP™ (Luminex Corp., Austin, Tex.) or BeadArray™ (Illumina, San Diego, Calif.) . The amount of a specific bead is quantitated by color that also exhibits fluorescence which indicates hybridization. The ratio of total hybridization events at differentially methylated sites versus other differentially methylated alleles (detected by simultaneous hybridization on the same system) determines the relative ratio of alleles, and hence the presence of aneuploidies.

### Example 4

### Detection of ERG Methylation Profile

Plasma Process: Maternal, fetal cord (from terminated 10-18 week umbilicus), and normal non-pregnant blood were collected in ACDA tubes, transferred to 15 ml conical tubes and spun for 10 minutes at 3000 rpm (1500 x g). The plasma layer above the RBC pellet was collected and transferred to a 15 ml conical tube, and re-spun at 1500×g, then frozen at −80° C. until DNA isolation.

DNA Extraction/Modification: DNA was extracted from the plasma using the QIAamp® DNA blood mini-kit (Qiagen, Hilden, Germany). DNA was bisulfite modified using the CpGenome™ Modification kit (Intergen Co., Purchase, N.Y.), according to manufacturer's protocol, and eluted in a final volume of 27 μl.

Nested PCR/Cloning: Flanking primers specific for a 396 bp region encompassing 21 potential CpG sites of the human ERG gene located on chromosome 21 within the Down's critical region (NCBI Reference Sequence No. NM 004449; GenBank Sequence Nos. Ml 7254; M21535) were designed and used in a PCR under standard conditions. Post PCR cleanup of the reaction was carried out using the QlAquick® PCR purification kit (Qiagen, Hilden, Germany). Nested primers were then used to further amplify the primary PCR product, then the resulting product was purified and cloned into TOPO vectors, transformed, and plated onto agar.

Plasmid Prep/Sequencing: A minimum of 25 positive colonies were picked from the plate for each sample type, grown 20 hours in 1X TB and the DNA extracted using a QLAprep® 96 Turbo Minikit (Qiagen, Hidel, Germany). Dye terminator sequencing of each clone was performed on an ABI PRISM® 7700 Sequence Detection System (Applied Biosystems, Foster City, Calif.). The resulting chromatograms were exported into Sequencher™ sequencing analysis software (Gene Codes Corp., Ann Arbor, Mich.) for final analysis.

Sequence Analysis: Methylation status was determined by analyzing the Sequence data for the presence of a cytosine (methylated) or thymidine (unmethylated) residue at the original CpG site. The resulting data was expressed in the number of methylated cytosine residues over the total number of clones sequenced to determine the percent methylation.

Primer Sequences:

| | |
|---|---|
| Primary sequences: | |
| 5′ TTAGTTGGTGAATTTTAGTATGG 3′ | (forward) |
| 5′ CCTTCCTCTCCTAACCTCA 3′ | (reverse) |
| Nested primers: | |
| 5′ GGTGAATTTTAGTATGG 3′ | (forward) |
| 5′ GAGGTTGAGGTTGATGTAGTG 3′ | (reverse) |

ERG Methylation Profile:

| CpG | % Methy- | | | | | | | | | | | |
|---|---|---|---|---|---|---|---|---|---|---|---|---|
| site No. | lated | 1 | 2 | 3 | 4 | 5 | 6 | 7 | 8 | 9 | 10 | 11 |

| | | | | | | | | | | | |
|---|---|---|---|---|---|---|---|---|---|---|---|
| Nor-1 | 100 | 100 | 100 | 100 | 100 | 100 | 100 | 100 | 100 | 100 | 100 |
| Nor-2 | 100 | 100 | 100 | 100 | 13 | 95 | 100 | 100 | 100 | 100 | 100 |
| Plac-1 | 81 | 52 | 40 | 55 | 38 | 52 | 60 | 60 | 83 | 74 | 74 |
| Plac-2 | 87 | 84 | 65 | 74 | 58 | 65 | 61 | 77 | 77 | 74 | 77 |
| Mat-1 | 100 | 82 | 100 | 100 | 100 | 93 | 78 | 98 | 100 | 100 | 100 |
| Mat-2 | 100 | 98 | 98 | 98 | 100 | 100 | 100 | 93 | 100 | 100 | 100 |

| | | | | | | | | | | | |
|---|---|---|---|---|---|---|---|---|---|---|---|
| CpG | % Methy- | | | | | | | | | | |
| site No. | lated | 12 | 13 | 14 | 15 | 16 | 17 | 18 | 19 | 20 | 21 |

| | | | | | | | | | | |
|---|---|---|---|---|---|---|---|---|---|---|
| Nor-1 | 100 | 100 | 100 | 100 | 100 | 100 | 100 | 100 | 100 | 100 |
| Nor-2 | 100 | 100 | 100 | 100 | 100 | 100 | 100 | 100 | 100 | 100 |
| Plac-1 | 71 | 76 | 55 | 81 | 74 | 74 | 52 | 69 | 57 | 43 |
| Plac-2 | 68 | 74 | 74 | 81 | 68 | 84 | 58 | 61 | 77 | 55 |
| Mat-1 | 91 | 96 | 84 | 91 | 100 | 100 | 100 | 98 | 100 | 98 |
| Mat-2 | 80 | 91 | 91 | 91 | 89 | 100 | 100 | 100 | 87 | 93 |

(Nor = Normal non-pregnant plasma/Plac = Placenta Tissue/Mat = Maternal plasma)

The total amount of circulating DNA present in maternal plasma samples was determined by quantitative real-time PCR assay for the glyceraldeyhde-3-phosphate dehydrogenase gene (GAPDH), which is present in all genomes. (Zhong, X. Y. et al., (2001) Am. J. Obstet. Gynecol. 184:414-419).

| | |
|---|---|
| Primers: | |
| 5′ CCCCACACACATGCACTTACC 3′ | (forward) |
| 5′ CCTAGTCCCAGGGCTTTGATT 3′ | (reverse) |
| Probe: | |
| 5′ AAAGAGCTAGGAAGGACAGGCAACTTGGC 3′ | |

DNA Recoveries from Whole Blood:

| | | Total DNA | | Fetal DNA | | |
|---|---|---|---|---|---|---|
| | Plasma | recovered | | Recovered | | |
| | Volume | (GAPDH) | | (FCY) | Total | Percent |
| Sample | Processed | ng | Total Copies | ng | Copies | Fetal |
| Maternal-1 | 5 | 126 | 38,181 | 0.576 | 87 | 0.50 |
| Maternal-2 | 5 | 106.2 | 32,182 | 9.09 | 1,377 | 8.5 |

### Example 5

### Detection of Male Fetal DNA in Maternal Plasma

Plasma Processing: Blood samples are collected in ACDA tubes, transferred to 15 ml conical tubes and spun for 10 minutes at 3000 rpm (1500×g). The plasma layer above the RBC pellet is collected transferred to a 15 ml conical tube, re-spun at 1500×g,, plasma above debris pellet is transferred to a fresh 50 ml conical tube and frozen at −80° C. until DNA isolation.

DNA Extraction/Bisulfite Modification: DNA was extracted from the plasma using the QIAamp® DNA blood mini-kit (Qiagen, Hilden, Germany). DNA was bisulfite modified using the CpGenome DNA Modification kit (Intergen Co., Purchase, N.Y.), according to manufacturer's protocol, and eluted in a final volume of 27 μl.

Post-conversion MS-FCY Quantitation: Maternal plasma with female and male fetus, normal plasma and female genomic source DNA samples were quantitated for FCY using the standard non-MS PCR prior to bisulfite conversion to determine the quantity of male DNA being introduced into the bisulfite treatment. The resulting data represents the amount of genome equivalents detected per TaqMan assay, pre and post bisulfite conversion to determine recovery efficiencies.

Real Time PCR (TaqMan) Assay Design: To detect Male DNA for quantitating sequences from the Y chromosome region, DYSI (NCBI Reference Sequence No. S86117) the following primers and probes were used. For purposes of this Example, FCY designates non-bisulfite converted DNA and MS-FCY designates methylation specific post-bisulfite converted DNA.

Forward Primers:

| | |
|---|---|
| FCY-F: | 5′ TCCTGCTTATCCAAATTCACCAT 3′ |
| MS-FCY-F | 5′ TTTAGGTATTTTTTGTTTATTTAAATTTATTAT 3′ |

Reverse Primers:

| | |
|---|---|
| FCY-R | 5′ ACTTCCCTCTGACATTACCTGATAATTG 3′ |
| MS-FCY-R | 5′ CATTTTACTTCCCTCTAACATTACC 3′ |

TaqMan Probes:

| | |
|---|---|
| FCY-P | 5′ AAGTCGCCACTGGATATCAGTTCCCTTGT 3′ |
| MS-FCY-P | 5′ AACTAATATCCAATAAC 3′ |

Amplicon Size:

| | |
|---|---|
| FCY | 85 bp |
| MS-FCY | 102 bp |

TaqMan Samples/Controls: Both normal non-pregnant and maternal with a female fetus samples were used as the plasma source DNA negative controls. Additional DNA from normal non-pregnant female PBMCs was used a negative control representing the genomic DNA source. Maternal plasma from male confirmed fetus was used as positive controls. CpGenome™ Universal Methylated DNA—male (Intergen Co., Purchase, N.Y.) was used for the standard curve, no template control was used as the blank. Sample designations: ML: Normal female non-pregnant plasma DNA; 50-E-1: Normal female non-pregnant genomic DNA; 50-E-2: Normal female non-pregnant genomic DNA; 23341-2: Maternal Plasma Male Fetus; 23343-2: Maternal Plasma Male Fetus; 23324-1: Maternal Plasma Female Fetus; 23324-2: Maternal Plasma Female Fetus. Results of the TaqMan assay are represented below.

| | Genome Equivalents (GE) | Genome Equivalents (GE) |
|---|---|---|
| | Male DNA | Male DNA |
| Sample | Pre Bisulfite Treatment | Post Bisulfite Treatment |
| ML | 0 | 0 |
| 50-E-1 | 0 | 0 |
| 50-E-2 | 0 | 0 |
| 23324-1 | 0 | 0 |
| 23324-2 | 0 | 0 |
| 23341-2 | 155 | 193 |
| 23343-2 | 286 | 154 |

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method of detecting fetal aneuploidies comprising
a) treating DNA isolated from maternal serum with a reagent that differentially modifies methylated and non-methylated DNA;
b) performing quantitative PCR with a first primer pair on a potentially aneuploid chromosome;
c) performing a control quantitative PCR with a second primer pair on a non-aneuploid chromosome; and
d) determining the ratio of the quantity of the two PCR products, thereby detecting fetal aneuploidies.

2. A method for detecting fetal chromosome aneuploidies comprising:
a) treating DNA isolated from maternal serum with bisulfite;
b) performing quantitative PCR on the sample with a primer pair homologous to a test chromosome sequence that is differentially methylated in maternal DNA and in fetal DNA, where the primer pair only primes bisulfite treated unmethylated DNA;
c) performing a control quantitative PCR with a primer pair homologous to a control chromosome sequence that is differentially methylated in maternal DNA and in fetal DNA, where the primer pair only primes bisulfite treated unmethylated DNA; and
d) determining the ratio of the quantity of PCR product produced for the test chromosome compared with the control chromosome.

3. A method for detecting alleles of a gene of interest in fetal DNA comprising:
a) treating DNA isolated from maternal serum with bisulfite;
b) performing PCR with a primer pair that amplifies the gene of interest of step b); and
c) analyzing the resulting PCR product to identify the allele of the gene of interest.

4. A method for detecting imprinted genes in a subject comprising:
a) treating DNA isolated from a subject with bisulfite;
b) performing PCR with a primer pair for a polymorphic region that only amplified bisulfite treated unmethylated DNA; and
c) analyzing the PCR product to identify the polymorphism thereby detecting imprinted genes in a subject.
